(19) United States
(12) Patent Application Publication
(10) Pub. No.: US 2024/0112815 A1
(43) Pub. Date: Apr. 4, 2024

(54) PROPOSAL SYSTEM AND PROPOSAL METHOD OF PROVIDING PROPOSAL FOR REDUCING RISK

(71) Applicant: Hitachi, Ltd., Tokyo (JP)

(72) Inventors: Giada CONFORTOLA, Tokyo (JP); Mika TAKATA, Tokyo (JP); Toshihiko KASHIYAMA, Tokyo (JP)

(21) Appl. No.: 18/465,001

(22) Filed: Sep. 11, 2023

(30) Foreign Application Priority Data
Sep. 29, 2022 (JP) 2022-156843

(51) Int. Cl. G16H 50/30

(52) U.S. Cl. CPC G16H 50/30

(57) ABSTRACT

A system is configured to acquire, from history data, entity data similar to target entity data which is data including a feature value of each of plurality of features related to a target entity. The history data includes entity data for each of a plurality of entities at each past time point. For each entity, entity data at a past time point includes a feature value at the time point for each feature of the entity. The system is configured to create, for each controllable feature among the plurality of features related to the target entity, based on statistics of a plurality of feature values in related case data which is data including entity data similar to the target entity data, a proposal of a feature value change for reducing a risk predicted by inputting the target entity data to a prediction model. A user interface which displays the proposal.

### BACKGROUND OF THE INVENTION

### 1. Field of the Invention

The present invention generally relates to a data processing technique of proving a proposal for reducing a risk.

### 2. Description of Related Art

In recent years, while the health life and the work life have been extended, an increase in medical cost and nursing cost has become an issue due to a change in a population structure such as the declining birthrate and the aging population. Therefore, it is desired to predict a risk of occurrence and intensification of health, medical care, nursing care, or the like, and to propose appropriate measures. A technique disclosed in PTL 1 relates to prediction of a risk that may occur in the future or a proposal for reducing a risk. PTL 1 discloses a technique for proposing a method for reducing a weight of a patient in order to reduce the risk of a disease.

### CITATION LIST

### Patent Literature

PTL 1: JP2022-64113A

### SUMMARY OF THE INVENTION

PTL 1 describes a weight that is a factor that can be directly controlled by improving lifestyle habits, but does not disclose a processing method when a plurality of features are handled in which any feature including a plurality of features other than a weight is to be improved.

A proposal system is configured to acquire, from history data, entity data similar to target entity data which is data including a feature value of each of a plurality of features related to a target entity. The history data includes entity data for each of a plurality of entities at each past time point. For each entity, entity data at a past time point includes a feature value at the time point for each feature of the entity. The proposal system is configured to create, for each controllable feature among the plurality of features related to the target entity, based on statistics of a plurality of feature values in related case data which is data including entity data similar to the target entity data, a proposal of a feature value change for reducing a risk predicted by inputting the target entity data to a prediction model. The prediction model is a model in which data including a feature value of each of a plurality of features related to an entity is input and a risk is output. The proposal system is configured to provide a user interface (UI) which displays a proposal created for the target entity.

According to the invention, it is possible to provide a high probability of implementing risk reduction.

### BRIEF DESCRIPTION OF THE DRAWINGS

FIG. 1 shows a configuration example of a proposal system according to an embodiment;

FIG. 2 shows a configuration example of a feature table;

FIG. 3 shows a configuration example of a patient extraction table;

FIG. 4 shows a configuration example of a time constraint table set;

FIG. 5 shows a configuration example of a domain DB;

FIG. 6 shows an example of a flow of entire processing according to the embodiment.

FIG. 7 shows an example of a processing flow of S603 in FIG. 6;

FIG. 8 shows an example of a processing flow of S702 in FIG. 7;

FIG. 9 shows an outline example of creation of a basic proposal for one feature;

FIG. 10 shows an example of a time boundary;

FIG. 11 shows an example of a flow of proposal creation processing;

FIG. 12 shows an outline example of determination of a risk reduction target corresponding to a target desired by a user;

FIG. 13 shows an outline example of alternative proposal creation;

FIG. 14 shows an example of a generic proposal created to achieve a risk reduction target;

FIG. 15 shows an example of a flow of proposal creation processing for each domain;

FIG. 16 shows an example of a patient GUI;

FIG. 17 shows an example of a constraint setting GUI; and

FIG. 18 shows an example of an alternative proposal selection GUI.

### DESCRIPTION OF EMBODIMENTS

In the following description, an “interface device” may be one or more interface devices. The one or more interface devices may be at least one of the following.
An I/O interface device that is one or more input and output (I/O) interface devices. The I/O interface device is an interface device for at least one of an I/O device or a remote display computer. The I/O interface device for the display computer may be a communication interface device. At least one I/O device may be a user interface device, for example, any one of an input device such as a keyboard and a pointing device, and an output device such as a display device.
A communication interface device that is one or more communication interface devices. The one or more communication interface devices may be one or more communication interface devices of the same type (for example, may be one or more network interface cards (NICs)), or two or more communication interface devices of different types (for example, an NIC and a host bus adapter (HBA)).

In the following description, a “memory” may be one or more memory devices which is an example of one or more storage devices, and may be typically a main storage device. At least one memory device in the memory may be a volatile memory device or a non-volatile memory device.

In the following description, a “persistent storage device” may be one or more persistent storage devices which is an example of one or more storage devices. Typically, the persistent storage device may be a non-volatile storage device (for example, an auxiliary storage device), and specifically, for example, a hard disk drive (HDD), a solid state drive (SSD), a non-volatile memory express (NVME) drive, or a storage class memory (SCM).

In the following description, a “storage device” may be either the “memory” or the “persistent storage device”.

In the following description, a “processor” may be one Or more processor devices. Typically, at least one processor device may be a microprocessor device such as a central processing unit (CPU), and may also be a processor device of another type such as a graphics processing unit (GPU). At least one processor device may be a single-core processor device or a multi-core processor device. At least one processor device may be a processor core. At least one processor device may be a processor device in a broad sense, such as a circuit (for example, a field-programmable gate array (FPGA), a complex programmable logic device (CPLD), or an application specific integrated circuit (ASIC)) which is an aggregate of gate arrays in a hardware description language that performs a partial or entire processing.

In the following description, processing may be described using “program” as a subject, and since a program is executed by a processor to perform predetermined processing while appropriately using a storage device and/or an interface device, the subject of the processing also may be a processor (or a device or a system including the processor). The program may be installed in a device such as a computer from a program source. The program source may be, for example, a recording medium (for example, a non-transitory recording medium) readable by a program distribution server or a computer. In the following description, two or more programs may be implemented as one program, or one program may be implemented as two or more programs.

In the following description, an expression such as “xxx table” may be used to describe information obtained as an output with respect to an input. The information may be data of any structure (for example, structured data or unstructured data), or may be a training model as represented by a neural network, a genetic algorithm, or a random forest that generates an output with respect to an input. Therefore, “xxx table” can be referred to as “xxx information”. In the following description, a configuration of each table is an example, one table may be divided into two or more tables, and all or a part of the two or more tables may be one table.

Hereinafter, an embodiment of the invention will be described with reference to the drawings.

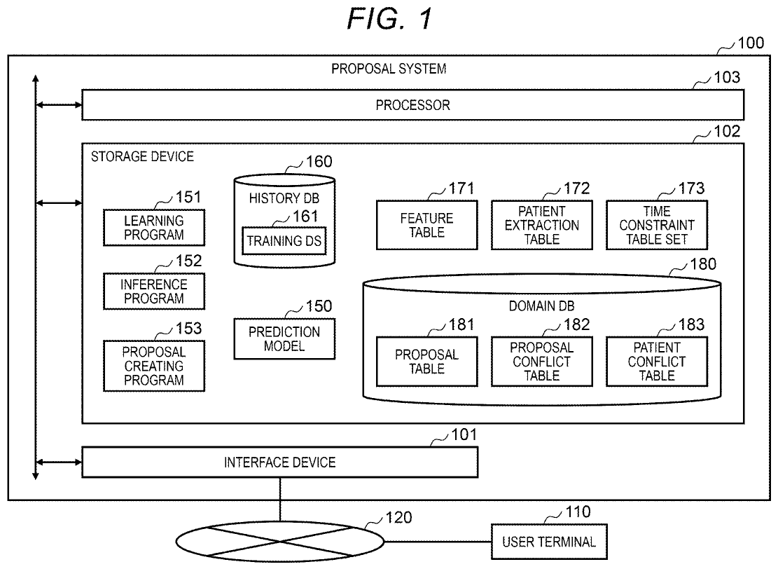

FIG. 1 shows a configuration example of a proposal system according to the embodiment.

A proposal system 100 according to the embodiment proposes a method of changing controllable feature data in order to reduce a risk predicted by a prediction model 150. The proposal system 100 estimates a time boundary and sets, based on the estimated time boundary, a risk reduction target that can be achieved within a designated time. The proposal system 100 creates proposals based on domain knowledge. The proposal system 100 filters (excludes) conflict proposals among the created proposals. In the embodiment, the “risk” means an occurrence probability of a defined event. For example, in relation to medical care, the risk may be a probability that an inpatient is hospitalized again, and in relation to manufacturing, the risk may be a probability of failure of a component. Hereinafter, as an example, it is assumed that the risk is a probability that a patient who is currently hospitalized is hospitalized again within 30 days when the patient is discharged from hospital.

The proposal system 100 may be an information processing terminal such as a personal computer, a physical computer system including one or more physical computers, or a logical computer system (for example, a cloud computing system) based on a physical computer system. The proposal system 100 includes an interface device 101, a storage device 102, and a processor 103 connected thereto.

The interface device 101 may communicate with a user terminal 110 via, for example, a communication network 120. The proposal system 100 may be a server system, and the user terminal 110 may be a client system. The user terminal 110 may be an information processing terminal such as a personal computer or a smartphone. The “user” typically refers to a doctor, but may be a nurse, a public health nurse, a medical therapist, or the like, and is not limited thereto. The user terminal 110 may be an example of an input and output console that is an input device (for example, a keyboard and a pointing device) and a display device.

The storage device 102 stores the prediction model 150. The prediction model 150 may be a machine learning model that inputs patient data and outputs data indicating a risk. The prediction model 150 may be a neural network or a gradient boosting model. Both (or one of) the risk and the time may be objective variables, and a plurality of features may be explanatory variables.

The storage device 102 stores data such as a history DB (database) 160, a feature table 171, a patient extraction table 172, a time constraint table set 173, and a domain DB 180. The history DB 160 includes data related to past health of each patient (specifically, data including past feature values for a plurality of features of each patient), and a part of the data and/or data different from the data are used as a training DS (data set) 161 of the prediction model 150.

The domain DB 180 includes a proposal table 181, a proposal conflict table 182, and a patient conflict table 183.

The storage device 102 stores computer programs such as a learning program 151, an inference program 152, and a proposal creating program 153. The learning program 151 performs learning of the prediction model 150 using the training DS 161. The inference program 152 predicts (infers) the risk of the patient by inputting the patient data to the trained prediction model 150. The proposal creating program 153 proposes a method for reducing the predicted risk. The proposal creating program 153 can receive a risk reduction target desired by the user from the user terminal 110.

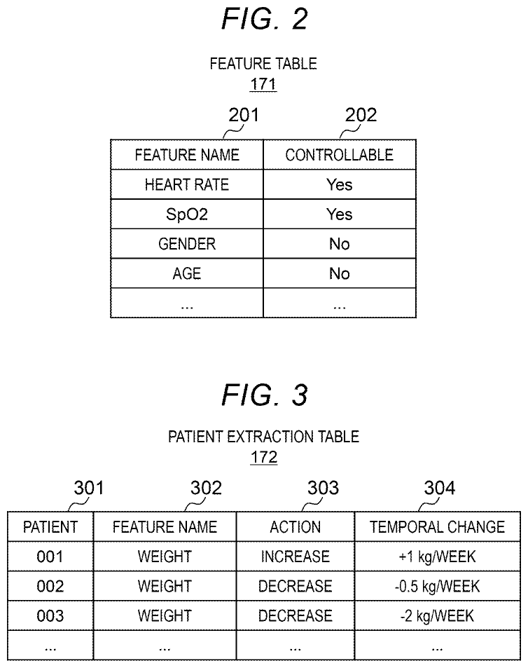

FIG. 2 shows a configuration example of the feature table 171.

The feature table 171 indicates whether each of the plurality of features of the patient is controllable. The feature table 171 has an entry for each feature. The entry includes data such as a feature name 201 and controllable 202. The feature name 201 indicates a name of the feature. The controllable 202 indicates whether the feature is controllable. The feature table 171 may be a list of feature names of controllable features.

FIG. 3 shows a configuration example of the patient extraction table 172.

The patient extraction table 172 may be generated based on data extracted from the history DB 160, and indicates how the feature data of the patient changes with time. The patient extraction table 172 has an entry for each set of a patient and a feature. The entry includes data such as a patient ID 301, a feature name 302, an action 303, and a temporal change 304.

The patient ID 301 indicates an ID of the patient. The feature name 302 represents a name of the feature of the patient. The action 303 indicates whether a value of the feature is an increase or a decrease. The temporal change 304 indicates a change per unit time in the value of the feature.

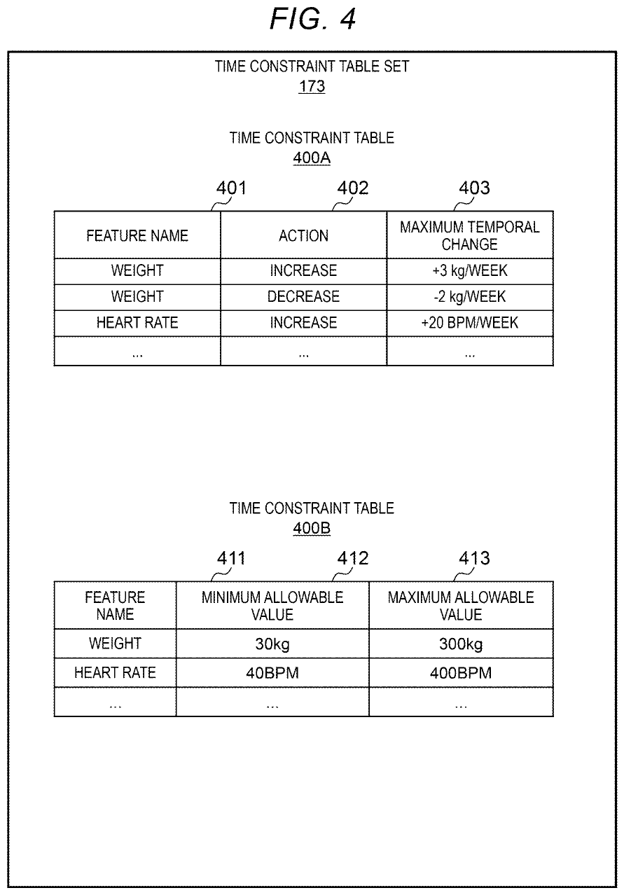

FIG. 4 shows a configuration example of the time constraint table set 173.

The time constraint table set 173 includes a first time constraint table 400A and a second time constraint table 400B.

The first time constraint table 400A indicates a possible change amount per unit time for each feature of the patient. The first time constraint table 400A has an entry for each feature. The entry includes data such as a feature name 401, an action 402, and a maximum temporal change 403. The feature name 401 indicates a name of the feature. The action 402 indicates whether a value of the feature is an increase or a decrease. The maximum temporal change 403 indicates an implementable maximum temporal change.

The second time constraint table 400B indicates a minimum value and a maximum value for each feature of the patient. The second time constraint table 400B has an entry for each feature. The entry includes data such as a feature name 411, a minimum allowable value 412, and a maximum allowable value 413. The feature name 411 indicates a name of the feature. The minimum allowable value 412 indicates a minimum feature value allowable for the feature. The maximum allowable value 413 indicates a maximum feature value allowable for the feature.

The time constraint table set 173 may be created by a user (for example, a doctor). For example, the time constraint table set 173 is a table received from the user terminal 110. The time constraint table set 173 may be prepared for each patient.

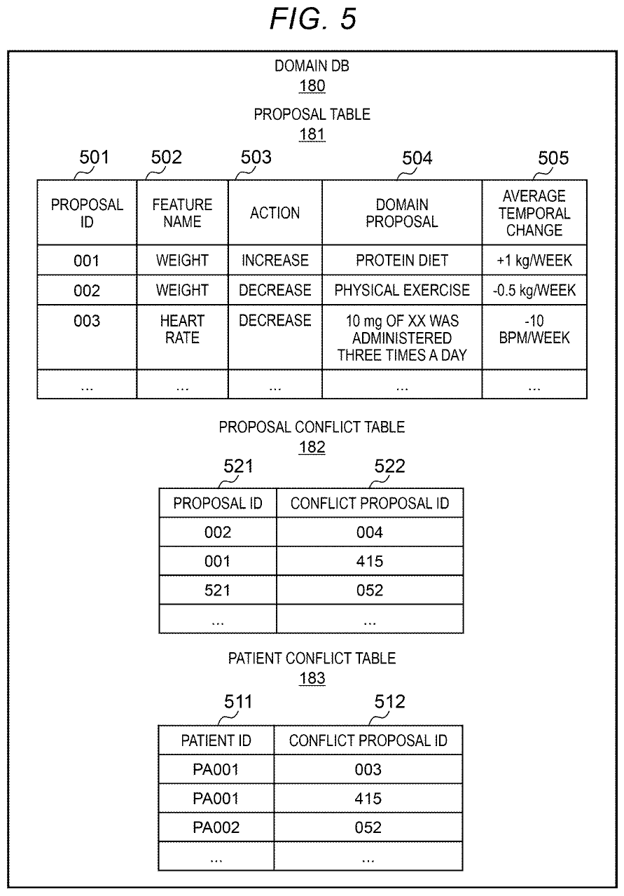

FIG. 5 shows a configuration example of the domain DB 180.

The domain DB 180 may be created by a user (for example, a doctor). As described above, the domain DB 180 includes a proposal table 181, a proposal conflict table 182, and a patient conflict table 183.

The proposal table 181 has an entry for each proposal. The entry includes data such as a proposal ID 501, a feature name 502, an action 503, a domain proposal 504, and an average temporal change 505. The proposal ID 501 indicates an ID of a domain proposal. The feature name 502 indicates a name of the feature. The action 503 indicates whether a value of the feature is an increase or a decrease. The domain proposal 504 indicates a detailed proposal. The average temporal change 505 indicates an average value of changes per unit time in the value of the feature.

The proposal conflict table 182 indicates a conflict between domain proposals. The proposal conflict table 182 has an entry for each set of a domain proposal and a domain proposal having a conflict. The entry includes data such as a proposal ID 521 and a conflict proposal ID 522. The proposal ID 521 indicates an ID of a domain proposal, and the conflict proposal ID 522 indicates an ID of a domain proposal that conflicts with the domain proposal. For example, it is assumed that there is a conflict in a domain proposal of “exercise” with respect to a domain proposal of “rest”, and the IDs of the domain proposals are set in the same entry.

The patient conflict table 183 indicates a conflict between a patient and a domain proposal. The patient conflict table 183 has an entry for each set of a patient and a domain proposal having a conflict. The entry includes data such as a patient ID 511 and a conflict proposal ID 512. The patient ID 511 indicates an ID of a patient. The conflict proposal ID 512 indicates an ID of a domain proposal that conflicts with the patient. For example, it is assumed that there is a conflict between a patient who is allergic to a certain drug and a domain proposal of taking the drug, and the IDs of the patients and the IDs of the domain proposals are set in the same entry.

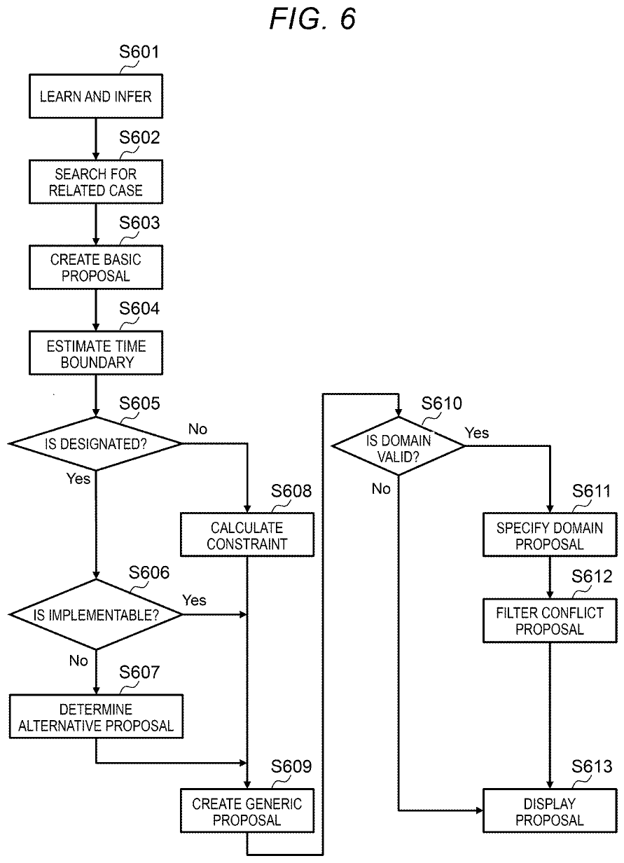

FIG. 6 shows an example of a flow of entire processing according to the embodiment.

In S601, the learning program 151 trains the prediction model 150 using the training DS 161. The inference program 152 inputs patient data of a target patient to the trained prediction model 150, thereby predicting a risk of the patient (a probability that the patient who is currently hospitalized is hospitalized again within 30 days when the patient is discharged from hospital). The training DS 161 may include, for example, for each of a plurality of patients, data indicating one or a plurality of dates and times, data including a value of each of a plurality of features related to the patient at each date and time, and data indicating the date and time of the re-hospitalization when the patient is hospitalized again. The history DB 160 may be used as the training DS 161. The plurality of features related to the patient may be a plurality of attribute items related to the patient, and may include, for example, personal attribute items such as age, gender, and blood type, and vital parameter items such as a heart rate and an SpO₂ measured during hospitalization. The patient data (patient data of the target patient) input to the trained prediction model 150 may include a value at the current time point for each of the plurality of features related to the target patient.

In S602, based on the patient data of the target patient, the proposal creating program 153 searches the history DB 160 for related case data that is data indicating cases related to the patient data of the target patient. For example, the history DB 160 may include, for each patient, data including, for each of a plurality of past time points, a feature value at that time point for each feature. The related case data may be patient data including a feature value of each feature for each patient other than the target patient at each of one or more past time points. That is, the related case data may include patient data for each of one or more patients other than the target patient at each of one or more past time points. The patient data included in the related case data may be patient data similar to the patient data of the target patient (for example, feature values of one or a plurality of features), for example, patient data having a distance (for example, cosine similarity or Euclidean distance) to the patient data of the target patient equal to or less than a threshold. The related case data may include patient data having a feature value similar to the feature value of the target patient with respect to one or more features (or features excluding the one or more features) specified by using an explanation calculation method such as SHapley Additive exPlanations (SHAP). A different weight (for example, a coefficient of a feature as an explanatory variable) may be assigned depending on the feature. A method of searching for the related case data is not limited to this example.

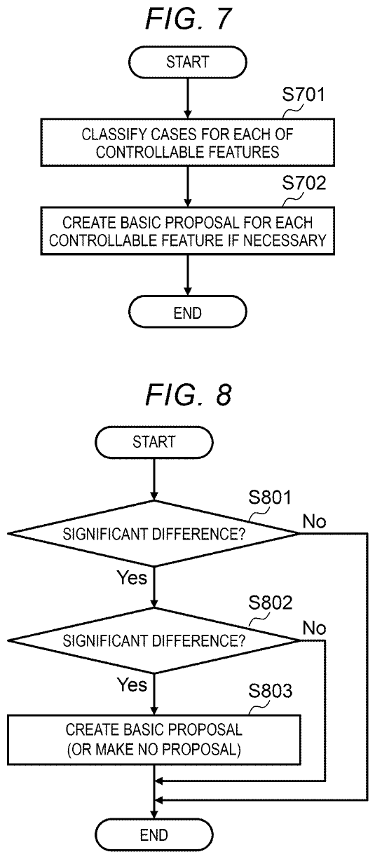

In S603, the proposal creating program 153 specifies one or more controllable features related to the target patient among a plurality of features related to the patient based on the extracted related case data and the feature table 171, and creates a basic proposal for each of the one or more features, which is a proposal to increase or decrease the feature value if the change in the feature value of the feature is necessary for reducing the risk. Details of S603 will be described later with reference to FIGS. 7 to 9.

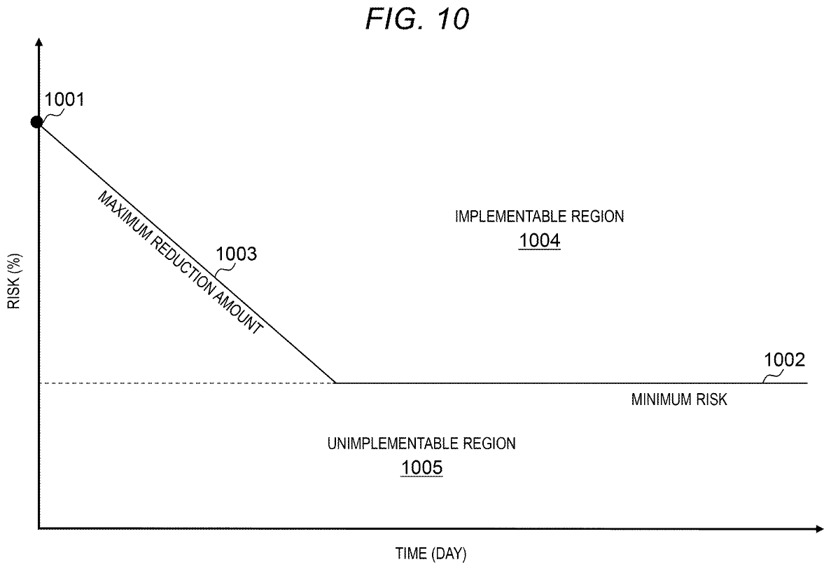

In S604, the proposal creating program 153 estimates the time boundary using at least one of the patient extraction table 172 and the time constraint table set 173. Details of S604 will be described later with reference to FIG. 10.

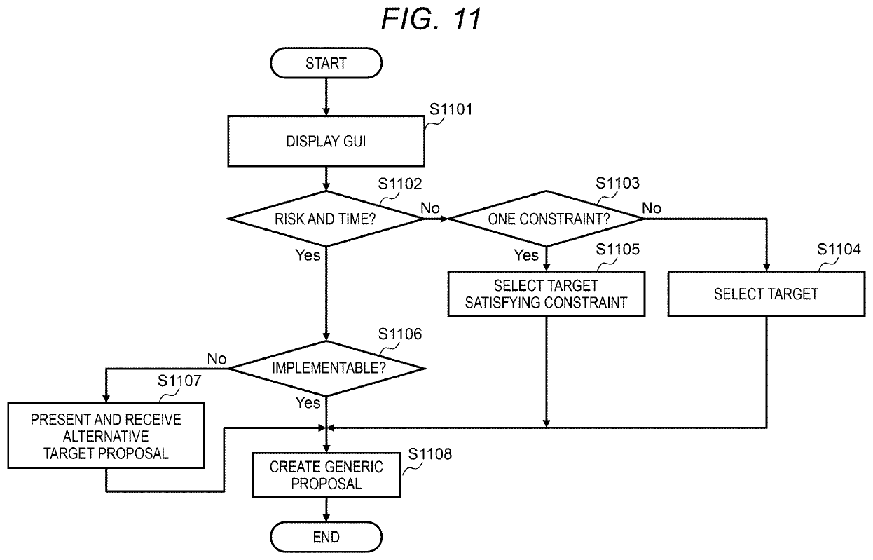

In S605, the proposal creating program 153 determines whether the constraint is designated by the user regarding at least one of the risk and the time. When the determination result of S605 is true (S605: Yes), the proposal creating program 153 determines whether the constraint of the user designation is implementable in S606. When the determination result of S606 is false (S606: No), the proposal creating program 153 determines an alternative proposal of the constraint (in other words, an alternative target proposal) in S607. When the determination result of S605 is false (S605: No), the proposal creating program 153 calculates the constraint in S608. When the determination result of S606 is true (S606: Yes), after S607 or after S608, the proposal creating program 153 creates a generic proposal as a quantitative proposal in S609. Details of S605 to S609 will be described later with reference to FIG. 11.

In S610, the proposal creating program 153 determines whether the domain DB 180 is valid. Whether the domain DB 180 is valid may be, for example, any one of the following.
When there is setting data indicating whether the domain DB 180 is valid, the data indicates whether the domain DB 180 is valid or invalid.
When the patient data of the target patient includes data indicating whether the domain DB 180 is used, the data indicates whether the domain DB 180 is used.
Whether there is data related to the target patient in the domain DB 180.

When the determination result of S610 is false (S610: No), in S613, the proposal creating program 153 provides patient proposal information (display information) including information indicating the generic proposal to the user terminal 110. Accordingly, a patient graphical user interface (GUI) displaying the patient proposal information is displayed on the user terminal 110. Instead of the GUI, another type of user interface (UI) may be provided.

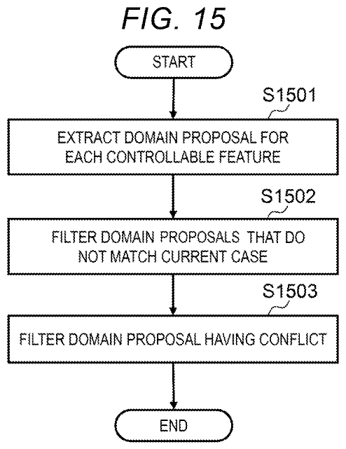

When the determination result of S610 is true (S610: Yes), in S611, the proposal creating program 153 specifies a domain proposal corresponding to the generic proposal of the target patient from the domain DB 180. In S612, the proposal creating program 153 checks whether there is a conflict in the specified domain proposal, and when there is a domain proposal having a conflict, the domain proposal is filtered (details of S611 to S612 will be described later with reference to FIG. 15). Thereafter, in S613, the proposal creating program 153 provides, to the user terminal 110, the patient proposal information (display information) including information indicating a generic proposal and a domain proposal not subjected to filtering among quantitative proposals. Accordingly, the patient GUI displaying the patient proposal information is displayed on the user terminal 110.

Hereinafter, the processing shown in FIG. 6 will be described in detail.

FIG. 7 shows an example of a processing flow of S603 in FIG. 6.

In S701, the proposal creating program 153 specifies controllable features based on the feature table 171. The proposal creating program 153 classifies feature cases indicated by the related case data into a low risk distribution and a high risk distribution for each of the specified controllable features.

Here, taking one feature as an example (“target feature” in the description of FIG. 7), the feature cases, the low risk distribution, and the high risk distribution are as follows.

The “feature cases” are a set of feature values of the target feature. That is, when the related case data includes the patient data of the plurality of patients and the patient data of each patient includes the feature values of the target features, the feature cases of the target features include the feature values of the target features in each patient data.

The “low risk distribution” is a distribution (for example, a normal distribution) of the feature values of the target features in the patient data corresponding to the low risk among the feature cases of the target features. The “patient data corresponding to the low risk” is patient data corresponding to a risk equal to or lower than the predicted risk for the target patient. The “risk corresponding to the patient data” may be a risk associated with the patient data (for example, a risk predicted in the past for the patient data, or a risk defined based on the predicted risk and an actual result (for example, the presence or absence of the re-hospitalization or the date and time of the re-hospitalization) indicated by the patient data), or may be a risk obtained by inputting the patient data to the prediction model 150.

The “high risk distribution” is a distribution (for example, a normal distribution) of the feature values of the target features in the patient data corresponding to the high risk among the feature cases of the target features. The “patient data corresponding to the high risk” is patient data corresponding to a risk higher than the predicted risk for the target patient.

The determination of the “low risk” and the “high risk” is not limited to the above-described method. For example, based on an entire risk average value, it may be determined whether the risk is equal to or larger than the average value or equal to or lower than the average value, or whether the risk is a low risk or a high risk.

In S702, the proposal creating program 153 determines a basic proposal for each controllable feature if necessary. The “basic proposal” is a proposal to increase or decrease the feature values. Specifically, the “basic proposal” is a proposal for bringing the feature values of the target features close to the low risk distribution (for example, a proposal for positioning the feature values of the target features in the low risk distribution).

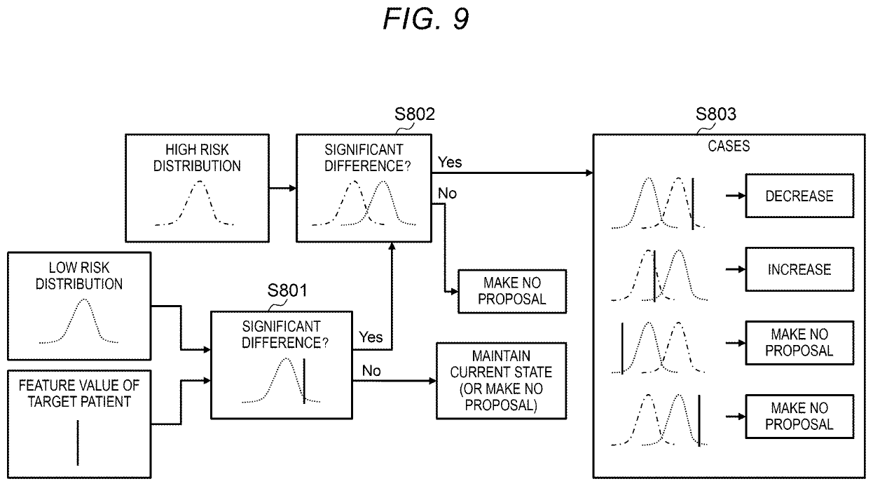

FIG. 8 shows an example of a processing flow of S702 in FIG. 7. FIG. 9 shows an outline example of creation of a basic proposal for one feature. One feature is taken as an example (“target feature” in the description of FIGS. 8 and 9). The feature value of the target feature of the target patient is referred to as a “current feature value” for convenience in the description of FIGS. 8 and 9.

In the low risk distribution and the high risk distribution, a horizontal axis represents a feature value, and a vertical axis represents a probability density.

In S801, the proposal creating program 153 determines whether there is a significant difference (for example, a difference satisfying a first condition) between the current feature value and the low risk distribution. For example, the proposal creating program 153 performs a statistical t-test in order to compare the current feature value with the low risk distribution. When the result of S801 is false (S801: No), the proposal creating program 153 maintains the current state (or makes no proposal). This is because the feature value of the target feature is already positioned in (or close to) the low risk distribution, and thus there is no need for a basic proposal.

When the result of S801 is true (S801: Yes), for example, when the significant difference is found at a certain reliability level, in S802, the proposal creating program 153 determines whether there is a significant difference (for example, a difference satisfying a second condition) between the low risk distribution and the high risk distribution. For example, the proposal creating program 153 performs a t-test to determine whether there is a significant difference between the low risk distribution and the high risk distribution. When the result of S802 is false (S802: No), the proposal creating program 153 makes no proposal. This is because the basic proposal that brings the feature value of the target feature close to the low risk distribution may also bring the feature value close to the high risk distribution.

When the result of S802 is true (S802: Yes), the proposal creating program 153 creates a basic proposal as necessary in S803. Specifically, for example, it is as follows. A third example and a fourth example among the following examples correspond to the example of S801: No, and thus may be not present in S803.

### A First Example

When the high risk distribution has a statistical value (for example, an average value) of the feature values larger than that of the low risk distribution and the current feature value is closer to the high risk distribution than the low risk distribution (for example, is positioned in the high risk distribution), the basic proposal is a proposal to decrease the current feature value. The basic proposal is to bring the current feature value close to the low risk distribution.

### A Second Example

When the high risk distribution has a statistical value of the feature value lower than that of the low risk distribution and the current feature value is closer to the high risk distribution than the low risk distribution (for example, is positioned in the high risk distribution), the basic proposal is a proposal to increase the current feature value. The basic proposal is to bring the current feature value close to the low risk distribution.

### The Third Example

When the high risk distribution has a statistical value of the feature value larger than that of the low risk distribution and the current feature value is closer to the low risk distribution than the high risk distribution (for example, is positioned in the low risk distribution), no basic proposal is made (or the basic proposal may be a proposal to maintain the current state).

### The Fourth Example

When the high risk distribution has a statistical value of the feature value lower than that of the low risk distribution and the current feature value is closer to the low risk distribution than the high risk distribution (for example, is positioned in the low risk distribution), no basic proposal is made (or the basic proposal may be a proposal to maintain the current state).

FIG. 10 shows an example of a time boundary.

The estimated time boundary can be expressed by an orthogonal coordinate system and represents a relationship between a time and a risk. An x-axis (an example of a first axis) corresponds to the time, and a y-axis (an example of a second axis orthogonal to the first axis) corresponds to the risk. A unit of the time is day in the example shown in FIG. 10, but is not limited thereto, and may be other units such as year, month, hour, minute, and second.

A current point 1001 on the vertical axis represents the risk of the target patient in a time 0, specifically, the risk predicted using the prediction model 150.

Orthogonal coordinates are divided into an implementable region 1004 and an unimplementable region 1005 by the time boundary. The time boundary is a boundary that divides a set of the risk and the time into an implementable set and an unimplementable set. Specifically, for example, the time boundary is formed of a first boundary portion 1002 and a second boundary portion 1003.

The first boundary portion 1002 represents a minimum risk of the target patient. The minimum risk of the target patient is a risk obtained based on the prediction model 150 when it is assumed that an optimum value is achieved for the controllable features of the target patient.

The second boundary portion 1003 represents a maximum reduction amount that is a maximum risk reduction amount per unit time. The maximum reduction amount is calculated by both or one of the following two methods (that is, calculated based on at least one of the patient extraction table 172 and the time constraint table set 173). In the following method, among the patient extraction table 172 and the time constraint table set 173, an entry corresponding to the controllable feature specified in processing of a previous stage may be referred to, and an entry corresponding to a feature not specified as the controllable feature in the processing of the previous stage may not be referred to.

The proposal creating program 153 calculates the maximum reduction amount based on the patient extraction table 172. For example, the proposal creating program 153 estimates a relationship between the predicted risk reduction amount and the time based on the feature names 302, the actions 303, and the temporal changes 304 of two or more patients, and the prediction model 150. The estimated relationship is the maximum reduction amount.
The proposal creating program 153 calculates the maximum reduction amount based on the time constraint table set 173. For example, the proposal creating program 153 calculates a maximum change amount for each controllable feature based on the maximum temporal change 403, the minimum allowable value 412, and the maximum allowable value 413, and estimates the relationship between the predicted risk reduction amount and the time based on the maximum change amount for each controllable feature and the prediction model 150. The estimated relationship is the maximum reduction amount.

FIG. 11 shows an example of a flow of proposal creation processing.

In step S1101, the proposal creating program 153 displays a GUI and waits for an input from the user. The GUI displayed here may display, for example, orthogonal coordinates expressing the estimated time boundary. The user may designate a desired portion on the orthogonal coordinates.

In S1102, the proposal creating program 153 determines whether constraints of both the risk and the time are designated. This determination may be, for example, a determination of whether a portion in the region on the orthogonal coordinate system shown in FIG. 10 is designated.

When the determination result of S1102 is false (S1102: No), in S1103, the proposal creating program 153 determines whether the constraint of one of the risk and the time is designated. This determination may be a determination of whether a portion on an axis of the orthogonal coordinate system is designated.

When the determination result of S1103 is false (S1103: No), in S1104, the proposal creating program 153 selects a risk reduction target for the target patient (for example, a target based on a past feature value time series related to the controllable feature of the target patient). The “risk reduction target” is a target indicating a time and a degree that the risk is to be reduced, that is, a set of the time and the risk reduction amount. In the orthogonal coordinate system of the time axis and the risk axis, a point corresponding to the risk reduction target is on the time boundary or on the implementable region 1004.

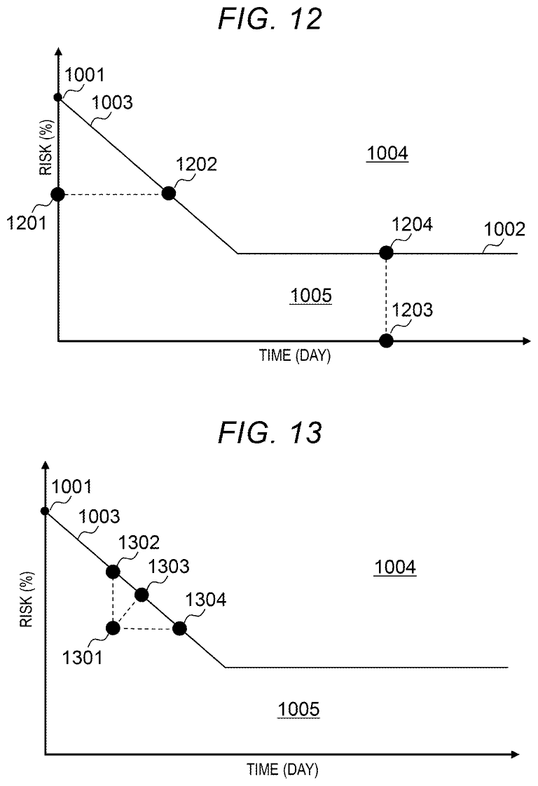

When the determination result of S1103 is true (S1103: Yes), the constraint designated by the user is a risk constraint or a time constraint. As shown in FIG. 12, the risk constraint is a constraint (in other words, a risk target) corresponding to a point 1201 on the risk axis, and the time constraint is a constraint (in other words, a time target) corresponding to a point 1203 on the time axis. In S1105, the proposal creating program 153 selects a risk reduction target satisfying the constraint designated by the user. Specifically, the following may be used.
When the constraint is the risk constraint, as shown in FIG. 12, the risk reduction target is a point 1202 that is a point on the time boundary or on the implementable region 1004 and has the same y-coordinate as the point 1201. According to the example shown in FIG. 12, the point 1202 is a point on the second boundary portion 1003. That is, the risk reduction target is a target corresponding to the shortest time among implementable times satisfying the risk constraint designated by the user.
When the constraint is the time constraint, as shown in FIG. 12, the risk reduction target is a point 1204 that is on the time boundary or on the implementable region 1004 and has the same x coordinate as the point 1203. According to the example shown in FIG. 12, the point 1204 is a point on the first boundary portion 1002. That is, the risk reduction target is a target corresponding to the minimum risk among implementable risks satisfying the time constraint designated by the user.

When the determination result of S1102 is true (S1102: Yes), in S1106, the proposal creating program 153 determines whether the constraint designated by the user corresponds to a point on the time boundary or on the implementable region 1004.

When the determination result of S1106 is false (S1106: No), in S1107, the proposal creating program 153 presents several alternative target proposals and receives a designation of an alternative target proposal desired by the user. For example, as shown in FIG. 13, it is assumed that the constraint designated by the user is a point 1301 on the unimplementable region 1005. The proposal creating program 153 calculates the following three alternative target proposals.
A first alternative target proposal is a target proposal corresponding to a point 1302 corresponding to minimum risk reduction among the risk reduction that can be achieved within a time designated by the user. The point 1302 may be a point on the implementable region 1004 on a straight line (straight line parallel to the y-axis) passing through the point 1301, instead of the point on the time boundary.
A second alternative target proposal is a target proposal corresponding to a point 1304 corresponding to a shortest time among the times at which the risk reduction designated by the user can be achieved. The point 1304 may be a point on the implementable region 1004 on a straight line (straight line parallel to the x-axis) passing through the point 1301, instead of the point on the time boundary.
A third alternative target proposal is a target proposal between the first alternative target proposal and the second alternative target proposal, specifically, for example, a target proposal corresponding to an intersection point 1303 between a perpendicular line passing through the point 1301 and the time boundary. The point 1303 may be a point on the implementable region 1004 on the perpendicular line passing through the point 1301, instead of the point on the time boundary.

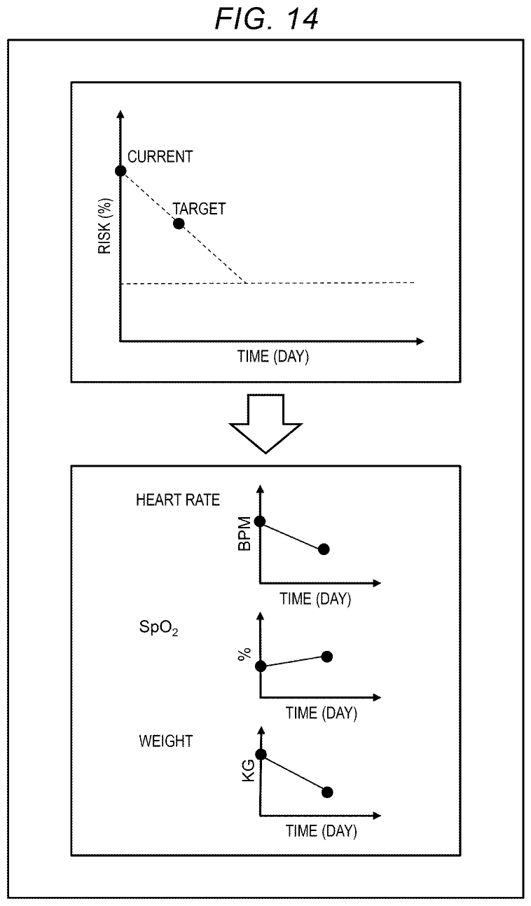

When the determination result of S1106 is true (S1106: Yes), or when S1107 is performed, the risk reduction target is determined. In S1108, the proposal creating program 153 creates a generic proposal for each controllable feature specified in the processing of the previous stage (S603 in FIG. 6) in order to achieve the determined risk reduction target by using, for example, the prediction model 150. As shown in FIG. 14, for each controllable feature, the generic proposal is a quantitative proposal as to how much the feature value is decreased or increased within the time represented by the risk reduction target (in a target time limit represented by the risk reduction target).

A correspondence relationship between FIG. 11 and FIG. 6 is, for example, as follows.
S1101 and S1102 in FIG. 11 correspond to S605 in
S1103 to S1105 in FIG. 11 correspond to S608 in FIG. 6.
S1106 in FIG. 11 corresponds to S606 in FIG. 6.
S1107 in FIG. 11 corresponds to S607 in FIG. 6.
S1108 in FIG. 11 corresponds to S1108 in FIG. 6.

FIG. 15 shows an example of a flow of proposal creation processing for each domain.

In S1501, the proposal creating program 153 extracts a domain proposal from the domain DB 180 for each controllable feature specified in the processing of the previous stage (S603 in FIG. 6). For example, an entry having the feature name 502 corresponding to the controllable feature is extracted from the proposal table 181 of the domain DB 180.

In S1502, the proposal creating program 153 filters domain proposals that do not match a current case (generic proposal created for the target patient) among the domain proposals extracted in S1501. For example, the following corresponding domain proposals are filtered.
A domain proposal having a feature name that does not match the feature names of all generic proposals.
A domain proposal that has a feature name that matches a feature name of any generic proposal but has an action that does not match an action (decrease or increase in the feature value) of the generic proposal.

In S1503, the proposal creating program 153 checks, for the domain proposals remaining in S1502, the presence or absence of a conflict between the domain proposals and the presence or absence of a conflict between the target patient and the domain proposals, and filters the domain proposal having a conflict. For example, the following is performed.
The proposal creating program 153 determines whether the conflict proposal ID 512 corresponding to the patient ID of the target patient is present in the patient conflict table 183. When the determination result is true, when there is a domain proposal identified from the conflict proposal ID 512, the proposal creating program 153 filters the domain proposal.
The proposal creating program 153 determines whether the conflict proposal ID 522 corresponding to the proposal ID of the domain proposal is present in the proposal conflict table 182. When the determination result is true, when there is a domain proposal identified from the conflict proposal ID 522, the proposal creating program 153 filters the domain proposal.

A correspondence relationship between FIG. 15 and FIG. 6 is, for example, as follows.
S1501 and 1502 in FIG. 15 correspond to S611 in
S1503 in FIG. 15 corresponds to S612 in FIG. 6.

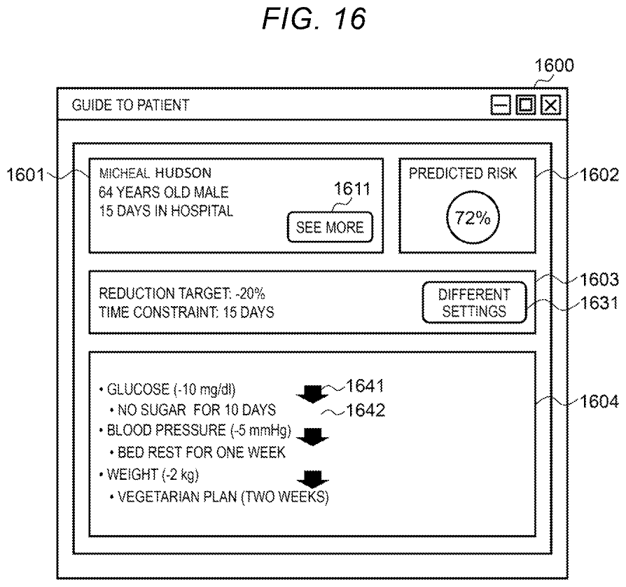

FIG. 16 shows an example of the patient GUI.

In FIG. 16, a patient GUI 1600 displays patient general information 1601, predicted risk information 1602, risk reduction target information 1603, and proposal information 1604.

The patient general information 1601 includes information such as a name, age, gender, and a period of hospitalization of the target patient. The patient general information 1601 may include feature values of uncontrollable features of the target patient. When a button 1611 is pressed, more detailed information of the target patient may be acquired from the history DB 160 and/or another DB, and displayed.

The predicted risk information 1602 indicates a risk predicted for the target patient using the prediction model 150.

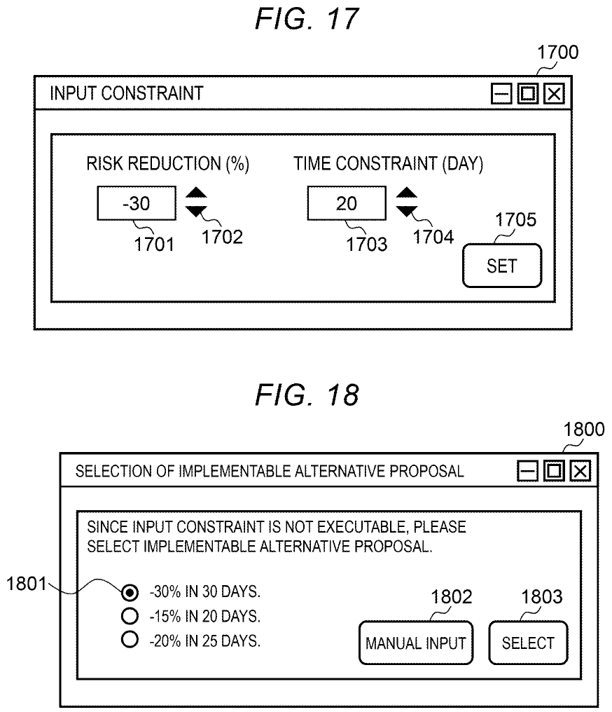

The risk reduction target information 1603 indicates a risk reduction target, that is, constraints on a risk and a time. When a button 1631 is pressed, the proposal creating program 153 displays a constraint setting GUI 1700 shown in FIG. 17 (details will be described later).

The proposal information 1604 indicates, for each controllable feature, a proposal of a method for achieving the risk reduction target indicated by the risk reduction target information 1603. The proposal is at least one of a generic proposal 1641 and a domain proposal 1642. In the proposal information 1604, an arrow indicates an action for each controllable feature. A downward arrow indicates a decrease in the feature value, and although not shown, an upward arrow indicates an increase in the feature value.

FIG. 17 shows an example of the constraint setting GUI 1700.

The constraint setting GUI 1700 includes GUI components (for example, a text box 1701 and an arrow button 1702) for inputting risk constraints and GUI components (for example, a text box 1703 and an arrow button 1704) for inputting time constraints. The constraint setting GUI 1700 receives inputs of a risk constraint and a time constraint. When a button 1705 is pressed after at least one of the risk constraint and the time constraint is input, the processing after S1102 in FIG. 11 may be performed, and the display may transition to the patient GUI 1600 shown in FIG. 16. The constraint setting GUI 1700 may display an orthogonal coordinate system indicating a time boundary, and a constraint (target) desired by the user may be input via the orthogonal coordinate system.

FIG. 18 shows an example of an alternative proposal selection GUI.

An alternative proposal selection GUI 1800 is displayed, for example, when the constraint input via the constraint setting GUI 1700 corresponds to an unimplementable region 1500. That is, the alternative proposal selection GUI 1800 selectably displays the calculated first to third alternative target proposals (the three alternative target proposals described with reference to FIG. 13).

When a radio button corresponding to any one of the alternative target proposals is designated and a button 1803 is pressed, the patient GUI 1600 shown in FIG. 16 is displayed, and the risk reduction target information 1603 of the patient GUI 1600 is information indicating the selected alternative target proposal. When a button 1802 is pressed, the alternative target proposal may be manually input.

For example, when information such as the name of the target patient is designated, the patient GUI 1600 in which display fields of the risk reduction target information 1603 and the proposal information 1604 are blank may be displayed, or any risk reduction target (for example, a target selected in S1104 of FIG. 11) related to the target patient and a proposal (for example, a proposal obtained in S1108 of FIG. 11 or FIG. 15) specified for the risk reduction target may be displayed on the patient GUI 1600. For example, when a constraint is input via the constraint setting GUI 1700 or when an alternative target proposal is selected or input via the alternative proposal selection GUI 1800, the risk reduction target information 1603 of the patient GUI 1600 may be updated, and the proposal information 1604 may be updated to information indicating a proposal of a method for achieving the risk reduction target indicated by the updated risk reduction target information 1603.

Although one embodiment is described above, the embodiment is an example for describing the invention, and is not intended to limit the scope of the invention to the embodiment. The invention can be implemented in various other forms. For example, the risk is not limited to the probability of re-hospitalization of the patient, and may be another risk related to medical care or a risk related to a field other than medical care (for example, an occurrence probability of a failure of a product). That is, the risk may be an occurrence probability of a defined event. The entity is not limited to a patient, and may be a physical entity such as a product or equipment, or a logical entity such as a virtual machine. The user terminal includes an information processing terminal of a doctor and an information processing terminal of a patient. The domain DB 180 may be constructed by the information processing terminal of the doctor, and a GUI such as the patient GUI 1600 may be provided to one or both of the information processing terminal of the doctor and the information processing terminal of the patient.

The above description can be summarized, for example, as follows. The following summary may include supplementary description of the above description and description of modifications.

The proposal system 100 includes the interface device 101 communicably connected to the user terminal 110 (an example of an input and output console), the storage device 102 that stores the prediction model 150 and the history DB 160 (an example of history data), and the processor 103 connected to the interface device 101 and the storage device 102. The prediction model 150 is a model in which data including a feature value of each of a plurality of features related to a patient (an example of an entity) is input and a risk is output. The history DB 160 includes patient data (an example of entity data) at each past time point for each of the plurality of patients. For each patient, patient data at a past time point includes a feature value at the time point for each feature of the patient.

The processor 103 acquires, from the history DB 160, patient data similar to target patient data which is data including a feature value of each of a plurality of features related to a target patient (an example of a target entity). The processor 103 creates, for each controllable feature among the plurality of features related to the target patient, based on statistics of a plurality of feature values in related case data which is data including the patient data similar to the target patient data, a proposal of a feature value change for reducing a risk predicted by inputting the target patient data to the prediction model 150. The processor 130 provides, to the user terminal 110, the patient GUI 1600 (an example of a UI) that displays a proposal created for the target patient.

As described above, a proposal is provided as to how to change the feature value for each controllable feature of the prediction model 150 to reduce the predicted risk. Therefore, a high possibility of implementing risk reduction is provided. For example, it is possible to provide a proposal for reducing the predicted risk by performing statistical analysis using the related case data, by considering all controllable features that may affect the risk.

For each controllable feature related to the target patient, the processor 103 may classify the plurality of feature values in the related case data into the first distribution and the second distribution having a statistically higher risk value than that of the first distribution. When a relationship between a current feature value, which is a feature value in the target patient data, and the first distribution satisfies the first condition (for example, S801: Yes) and a relationship between the first distribution and the second distribution satisfies the second condition (for example, S802: Yes), the processor 103 may create a proposal as to whether to decrease or increase the current feature value, which is a proposal for bringing the current feature value close to the first distribution. Accordingly, it is possible to provide a proposal as to whether to decrease or increase the controllable feature value for reducing the risk without particularly requiring domain knowledge. An example of the first distribution may be the low risk distribution, and an example of the second distribution may be the high risk distribution. The low risk distribution may be a distribution of feature values in the patient data corresponding to the low risk (risk equal to or lower than the predicted risk for the target patient). The high risk distribution may be a distribution of feature values in the patient data corresponding to the high risk (risk higher than the predicted risk for the target patient).

The processor 103 may take the created proposal as a quantitative proposal representing a time constraint and an increase amount or a decrease amount of a feature value for each controllable feature related to the target patient, based on a time boundary which is a boundary dividing a set of a risk and a time into an implementable set and an unimplementable set, and a risk reduction target which is a target indicating a time and a degree that the risk is to be reduced. The patient GUI 1600 may display the quantitative proposal for each controllable feature related to the target patient. Accordingly, since a time and a method of changing a feature value is displayed in order to implement the risk reduction target for each controllable feature, it is easy to understand an action to be taken by the target patient, and the possibility of implementing the risk reduction increases. Since there is a time boundary, it is possible to provide, as the quantitative proposal, a proposal for implementing the risk reduction in the shortest time within a possible range or a proposal for implementing the maximum risk reduction amount in a possible range.

When designation of one constraint of a risk constraint which is a target of the risk and a time constraint which is a target of the time for the target patient is received (for example, S1103: Yes), the processor 103 may determine the other constraint of the risk constraint and the time constraint to be a constraint on the time boundary or a constraint in the implementable set. A set of the one constraint and the other constraint may be the risk reduction target. Accordingly, it is possible to set an appropriate risk reduction target in which the other constraint satisfying the designated one constraint within a possible range is defined.

When designation of both constraints of a risk constraint which is a target of the risk and a time constraint which is a target of the time for the target patient is received (for example, S1102: Yes), the processor 103 may determine whether a set of the risk constraint and the time constraint corresponds to any one of a constraint on the time boundary and a constraint in the implementable set. When the determination result is true (for example, S1106: Yes), the set of the designated risk constraint and time constraint may be the risk reduction target. Accordingly, it is possible to avoid setting an inappropriate risk reduction target that cannot be implemented.

On the other hand, when the determination result is false (for example, S1106: No), the processor 103 may create one or a plurality of alternative target proposals, provide, to the user terminal 110, the alternative proposal selection GUI 1800 displaying the one or the plurality of alternative target proposals, and receive designation of an alternative target proposal which is selection of any one of the one or the plurality of alternative target proposals (or manual input of the alternative target proposal). The alternative target proposal may be a target proposal obtained by changing one and/or both of the set of the risk constraint and the time constraint to correspond to any one of the constraint on the time boundary and the constraint in the implementable set. The designated alternative target proposal may be the risk reduction target. Accordingly, when an unimplementable target is designated, it is possible to provide an implementable alternative target proposal by changing at least one of the risk constraint and the time constraint. The created alternative target proposal may be at least one of a proposal in which the designated time constraint is maintained and the risk constraint is changed, a proposal in which the designated risk constraint is maintained and the time constraint is changed, and a proposal in which both the time constraint and the risk constraint are changed.

The storage device 102 may store the domain DB 180 (an example of the domain data). The domain DB 180 may include the proposal table 181 (an example of the proposal data). The proposal table 181 may include data representing a domain proposal which is a proposal of a specific method for implementing the action with respect to the feature, for each set of a feature and an action of decrease or increase of a feature value. When the proposal table 181 includes data representing a set of a feature and an action matching the set of the feature and the action in the created proposal, the processor 103 may specify a domain proposal corresponding to the set from the proposal table 181. When a proposal in which a domain proposal is specified is present in the created proposals, the patient GUI 1600 may display the specified domain proposal 1642. As described above, by adding the domain-based proposal as, for example, an option, it is possible to provide a proposal of a specific action for reducing the risk. For example, when the created proposals include a proposal for reducing a value of a heart rate of the target patient, it is expected to provide a domain proposal as to what drug is to be prescribed to reduce the heart rate.

The domain DB 180 may include, for each domain proposal, the proposal conflict table 182 (an example of the proposal conflict data) that is data representing a domain proposal that conflicts with the domain proposal. For each of the specified domain proposals, when a domain proposal conflicting with the domain proposal is specified from the specified domain proposals based on the proposal conflict table 182, the processor 103 may filter the conflict domain proposal. The patient GUI 1600 may display domain proposals that remain without being filtered. Accordingly, it is possible to avoid providing another domain proposal having a conflict with the domain proposal.

The domain DB 180 may include, for each patient, the patient conflict table 183 (an example of the entity conflict data) which is data representing a domain proposal that conflicts with the patient. When a domain proposal that conflicts with the target patient is specified from the specified domain proposals based on the patient conflict table 183, the processor 103 may filter the domain proposal having the conflict. The patient GUI 1600 may display domain proposals that remain without being filtered. Accordingly, it is possible to avoid providing a domain proposal having a conflict with the patient.

What is claimed is:

1. A proposal system comprising:
an interface device configured to be communicably connected to an input and output console;
a storage device configured to store a prediction model and history data; and
a processor configured to be connected to the interface device and the storage device, wherein
the prediction model is a model in which data including a feature value of each of a plurality of features related to an entity is input and a risk is output,
the history data includes entity data for each of a plurality of entities at each past time point,
for each entity, entity data at a past time point includes a feature value at the time point for each feature of the entity, and
the processor is configured to
acquire, from the history data, entity data similar to target entity data which is data including a feature value of each of a plurality of features related to a target entity,
create, for each controllable feature among the plurality of features related to the target entity, based on statistics of a plurality of feature values in related case data which is data including entity data similar to the target entity data, a proposal of a feature value change for reducing a risk predicted by inputting the target entity data to the prediction model, and
provide, to the input and output console, a user interface (UI) which displays a proposal created for the target entity.

2. The proposal system according to claim 1, wherein
for each controllable feature related to the target entity, the processor is configured to
classify the plurality of feature values in the related case data into a first distribution and a second distribution having a risk value statistically larger than that of the first distribution, and
create, when a relationship between the first distribution and a current feature value, which is a feature value in the target entity data, satisfies a first condition and a relationship between the first distribution and the second distribution satisfies a second condition, a proposal for bringing the current feature value close to the first distribution, the proposal as to whether to decrease or increase the current feature value.

3. The proposal system according to claim 1, wherein
the processor is configured to take the created proposal as a quantitative proposal representing a time constraint and an increase amount or a decrease amount of a feature value for each controllable feature related to the target entity, based on a time boundary which is a boundary dividing a set of a risk and a time into an implementable set and an unimplementable set, and a risk reduction target which is a target indicating a time and a degree that the risk is to be reduced, and
the UI is configured to display the quantitative proposal for each controllable feature related to the target entity.

4. The proposal system according to claim 3, wherein
when designation of one constraint of a risk constraint which is a target of the risk and a time constraint which is a target of the time for the target entity is received, the processor is configured to determine the other constraint of the risk constraint and the time constraint to be a constraint on the time boundary or a constraint in the implementable set, and
a set of the one constraint and the other constraint is the risk reduction target.

5. The proposal system according to claim 3, wherein
when designation of both constraints of a risk constraint which is a target of the risk and a time constraint which is a target of the time for the target entity is received, the processor is configured to determine whether a set of the risk constraint and the time constraint corresponds to any one of a constraint on the time boundary and a constraint in the implementable set, and
when the determination result is true, a set of the risk constraint and the time constraint is the risk reduction target.

6. The proposal system according to claim 5, wherein
when the determination result is false, the processor is configured to
create one or a plurality of alternative target proposals,
provide a UI displaying the one or the plurality of alternative target proposals to the input and output console, and
receive designation of an alternative target proposal which is selection of any one of the one or the plurality of alternative target proposals or manual input of the alternative target proposal,
the alternative target proposal is a target proposal obtained by changing one and/or both of the set of the risk constraint and the time constraint to correspond to any one of the constraint on the time boundary and the constraint in the implementable set, and
the designated alternative target proposal is the risk reduction target.

7. The proposal system according to claim 1, wherein
the storage device is configured to store domain data,
the domain data includes proposal data including data representing a domain proposal which is a proposal of a specific method for implementing the action with respect to the feature, for each set of the feature and an action of decrease or increase of the feature value,
when the proposal data includes data representing a set of a feature and an action matching a set of a feature and an action in the created proposal, the processor specifies a domain proposal corresponding to the set from the proposal data, and
when a proposal in which a domain proposal is specified is present in the created proposals, the UI displays the specified domain proposal.

8. The proposal system according to claim 7, wherein
the domain data includes, for each domain proposal, proposal conflict data which is data representing a domain proposal conflicting with the domain proposal,
for each of the specified domain proposals, when a domain proposal conflicting with the domain proposal is specified from the specified domain proposals based on the proposal conflict data, the processor is configured to filter the conflict domain proposal, and
the UI is configured to display domain proposals which remain without being filtered.

9. The proposal system according to claim 7, wherein
the domain data includes entity conflict data which is data representing a domain proposal conflicting with the entity for each entity,
when a domain proposal conflicting with the target entity is specified from the specified domain proposals based on the entity conflict data, the processor is configured to filter the conflict domain proposal, and
the UI is configured to display domain proposals which remain without being filtered.

10. A proposal method comprising:
acquiring by a computer, from history data, entity data similar to target entity data which is data including a feature value of each of a plurality of features related to a target entity;
the history data including entity data for each of a plurality of entities at each past time point,
for each entity, entity data at a past time point including a feature value at the time point for each feature of the entity,
creating by the computer, for each controllable feature among the plurality of features related to the target entity, based on statistics of a plurality of feature values in related case data which is data including entity data similar to the target entity data, a proposal of a feature value change for reducing a risk predicted by inputting the target entity data to a prediction model; and
the prediction model being a model in which data including a feature value of each of a plurality of features related to an entity is input and a risk is output,
providing by the computer a user interface (UI) which displays a proposal created for the target entity.

11. A computer program causing a computer to
acquire, from history data, entity data similar to target entity data which is data including a feature value of each of a plurality of features related to a target entity;
the history data including entity data for each of a plurality of entities at each past time point,
for each entity, entity data at a past time point including a feature value at the time point for each feature of the entity,
create, for each controllable feature among the plurality of features related to the target entity, based on statistics of a plurality of feature values in related case data which is data including entity data similar to the target entity data, a proposal of a feature value change for reducing a risk predicted by inputting the target entity data to a prediction model; and
the prediction model being a model in which data including a feature value of each of a plurality of features related to an entity is input and a risk is output,
provide a user interface (UI) which displays a proposal created for the target entity.
